(19) United States
(12) Patent Application Publication
(10) Pub. No.: US 2020/0318174 A1
(43) Pub. Date: Oct. 8, 2020

(54) COMPOSITIONS AND METHODS FOR IDENTIFYING AND CHARACTERIZING GENE TRANSLOCATIONS, REARRANGEMENTS AND INVERSIONS

(71) Applicant: Agilent Technologies, Inc., Santa Clara, CA (US)

(72) Inventors: Rosanne WELCHER, Ventura, CA (US); Mark VERARDO, Ventura, CA (US); Jurgen DEL FAVERO, Tienen (BE); Michael RUVOLO, San Diego, CA (US); Russell BALDOCCHI, Santa Barbara, CA (US); Lien HEYRMAN, Zwijndrecht (BE); Dirk GOOSSENS, Antwerp (BE)

(21) Appl. No.: 16/836,512

(22) Filed: Mar. 31, 2020

Related U.S. Application Data
(60) Provisional application No. 62/828,642, filed on Apr. 3, 2019.

(51) Int. Cl. C12Q 1/6841; C12Q 1/6874; C12Q 1/6886; C12N 15/10; G01N 21/64

(52) U.S. Cl. CPC C12Q 1/6841; C12Q 1/6874; C12Q 1/6886; G01N 2021/6439; G01N 21/6428; C12Q 2600/156; C12N 15/1003; C12N 15/1096

(57) ABSTRACT

In alternative embodiments, provided are methods comprising use of FISH, IHC or equivalent gene fusion detection protocols, and gene sequencing, wherein optionally the gene sequencing is high throughput or next generation gene sequencing, for the identification and characterization of gene abnormalities such as gene breakages; optionally gene breakages comprise gene translocation, gene rearrangements and/or gene inversions. In alternative embodiments, genes or transcripts are analyzed from individuals suspected of having cancer, and the analysis is carried out on biological samples taken from these individuals. This identification and characterization of gene abnormalities can be used in the diagnosis and treatment of a cancer.

### RELATED APPLICATIONS

This U.S. Utility Patent Application claims the benefit of priority under 35 U.S.C. § 119(e) of U.S. Provisional Application Ser. No. (U.S. Ser. No.) 62/828,642 filed Apr. 3, 2019. The aforementioned application is expressly incorporated herein by reference in its entirety and for all purposes.

### TECHNICAL FIELD

This invention generally relates to molecular biology and precision medicine, and cancer diagnostics, cancer screening and treatment. In alternative embodiments, provided are methods comprising use of in situ hybridization (ISH), such as fluorescent ISH (FISH), immunohistochemistry (IHC) or equivalent gene fusion detection protocols and gene sequencing, for example, high throughput or next generation gene sequencing, for the identification and characterization of gene abnormalities such as gene breakages, for example, gene translocation, gene rearrangements and/or gene inversions. In alternative embodiments, genes or transcripts are analyzed from individuals suspected of having cancer or another condition resulting from gene abnormalities, and the analysis is carried out on biological samples taken from these individuals. This identification and characterization of gene abnormalities can be used in the diagnosis and treatment of a cancer.

### BACKGROUND

Recurrent, non-random chromosomal translocations are associated with disease conditions such as cancers, for example, solid tumors or hematologic malignancies. Particular translocations can be associated with particular subtypes of cancer, such as subtypes of leukemia, thus aiding in their diagnosis, treatment and disease monitoring allowing personalized precision medicine.

Translocations involving the MLL gene are among the most common of these non-random translocations associated with hematologic malignancies. The presence of distinct MLL rearrangements is an independent dismal prognostic factor, while very few MLL rearrangements display either a good or intermediate outcome. There are a large number of different chromosomal loci that partner with MLL in these translocations, over 60 partner genes or regions have been identified. Leukemias with an MLL translocation include infant leukemia, therapy-related leukemia, acute myelogenous leukemia (AML), T-cell ALL, B lineage ALL, myelodysplastic syndrome (MDS), lymphoblastic lymphoma, and Burkitt's lymphoma.

Translocations involving the ALK (anaplastic lymphoma kinase, also known as ALK tyrosine kinase receptor or CD246; see Du X. et al, 2018) gene also are non-random translocations associated with hematologic malignancies. A small inversion in chromosome 2p can lead to an aberrant ALK gene translocation and presence of aberrant ALK protein in the cytoplasm, resulting in uncontrolled cellular proliferation and survival. ALK fusion protein-related cancers include anaplastic large-cell lymphomas, diffuse large B-cell lymphomas, systemic histiocytosis, inflammatory myofibroblastic tumors, esophageal squamous cell carcinomas and non-small-cell lung carcinomas.

### SUMMARY

In alternative embodiments, provided are methods for identifying and characterizing a gene breakage in a biological sample, wherein the gene breakage is characterized or identified by a nucleic acid sequencing only after the biological sample is positively identified as having a gene breakage by an ISH (in situ hybridization) or an IHC (immunohistochemistry) assay, or the gene breakage is characterized or identified by a nucleic acid sequencing only if the biological sample is not positively identified as not having a gene breakage, the method comprising: (a) providing or having provided a biological sample; (b) providing or having provided probes designed to detect the gene breakage in the gene or its corresponding transcript, wherein optionally the probes are designed as fusion-signal FISH or split-signal (also called “break-apart”) FISH probes; (c) detecting the presence or absence of a gene breakage in the biological sample using the ISH (in situ hybridization) or the IHC (immunohistochemistry) assay; (d) sequencing or having sequenced nucleic acid from the biological sample if a gene breakage is detected in the biological sample to characterize the gene or genes involved in the gene breakage, or sequencing or having sequenced nucleic acid from the biological sample if it is not positively determined that a gene breakage has not occurred in the biological sample; and if it is positively determined that no gene breakage has occurred (or is present) in the biological sample the sample is not sequenced or had sequenced; and (e) analyzing or having analyzed the sequence of the sequenced nucleic acid to identify or characterize the gene or genes involved in the gene breakage.

In alternative embodiments of methods as provided herein:
the gene breakage comprises a gene translocation, a gene rearrangement or a gene inversion;
the ISH is a Fluorescent ISH, or FISH assay;
the biological sample is or comprises a biopsy or a cell or tissue sample, or can be in the form of a bone marrow smear (optionally, a bone marrow aspirate smear), a cytological sample, a serum sample, a cytological blood sample, an aspirate sample, a liquid biopsy a, blood smear, a paraffin embedded tissue preparation, an enzymatically dissociated tissue sample, an uncultured bone marrow, an uncultured amniocyte and/or cytospin preparation, or a tissue culture preparation;
the detecting the presence or absence of a gene breakage in the biological sample comprises: counting the total number or percentage of cells having signals indicating the presence of a gene breakage (optionally these cells have discretely colored signals such as red and green FISH signals), and if the total number or percentage reaches a threshold, which can be empirically or logically determined for each gene of interest, then the samples are considered positive and sequenced;
the detecting the presence or absence of a gene breakage in the biological sample comprises: counting the total number or percentage of cells having signals indicating no gene breakage (optionally these cells have yellow in addition to discrete red and green FISH signals), and if the total number or percentage of gene breakage negative cells does not reach a threshold that positively indicates no gene breakage in the sample, which can be empirically or logically determined for each gene of interest, then the samples are considered positive and sequenced (sequencing is performed only on samples not definitely identified as negative for a gene breakage);
the sequencing of the nucleic acid from the biological sample comprises use of a high throughput or Next Generation Sequencing or massively parallel signature sequencing (or MPSS), and optionally the sequencing comprises first converting cell transcripts or mRNA to cDNA, where the cDNA is used for sequencing library preparation, and optionally the preparation of the cDNA is followed by an adaptor ligation, optionally comprising use of a multiplex PCR (polymerase chain reaction) (mPCR) step with primers that allow for a subsequent universal PCR step; and/or
the gene breakage is in an MLL (mixed lineage leukemia) gene or an ALK (anaplastic lymphoma kinase) gene.

[symbol] method for diagnosing a disease or condition in an individual comprising use of the method for identifying and characterizing a gene breakage in a biological sample as provided herein, wherein a specific gene breakage is associated with a particular disease or condition, and if the specific gene breakage is found to be present in a biological sample from the individual, then the individual can be diagnosed with that disease or condition.

In alternative embodiments, provided are methods for treating, ameliorating or preventing a disease or condition in an individual in need thereof, comprising use of the method for identifying and characterizing a gene breakage in a biological sample as provided herein, or a method as provided herein for diagnosing a disease or condition in an individual, wherein a specific gene breakage is associated with a particular disease or condition, and if the specific gene breakage is found to be present in a biological sample from the individual, then the individual can be diagnosed with that disease or condition and the individual is treated for that disease or condition. In alternative embodiments, a breakage is in an MLL (mixed lineage leukemia) gene, and the disease or condition diagnosed or treated is: an infant leukemia, a therapy-related leukemia, an acute myelogenous leukemia (AML), a T-cell ALL, a B lineage acute lymphoblastic leukemia (ALL), a myelodysplastic syndrome (MDS), a lymphoblastic lymphoma or Burkitt's lymphoma. In alternative embodiments, a breakage is in an ALK (anaplastic lymphoma kinase gene), and the disease or condition diagnosed or treated is: an anaplastic large-cell lymphoma, a diffuse large B-cell lymphoma, a systemic histiocytosis, an inflammatory myofibroblastic tumor, an esophageal squamous cell carcinoma or a non-small-cell lung carcinoma.

In alternative embodiments, provided are kits comprising components or materials for use in practicing a method as provided herein, wherein optionally the kit further comprises instructions for practicing a method as provided herein. In alternative embodiments, the components or materials for use in practicing a method as provided herein comprise: PCR (polymerase chain reaction) reagents and/or probes, the optionally the PCR is multiplex PCR (mPCR); probes and/or reagents for conducting ISH (in situ hybridization) or an IHC (immuno-histochemistry), wherein optionally the ISH is a fluorescent ISH, or FISH; and/or probes and/or reagents for conducting reverse transcription, or for converting RNA to a cDNA.

In alternative embodiments, provided are methods for assessing the gene breakage status of a gene of interest in a subject, wherein optionally the gene breakage is a gene translocation, a gene rearrangement or a gene inversion, the method comprising: (a) performing an in situ hybridization analysis or an immunohistochemistry analysis on a biological sample from said subject to determine whether said subject has a gene breakage in said gene of interest; and (b) if said in situ hybridization or immunohistochemistry analysis indicates that said subject has a gene breakage in said gene of interest or indicates that it is unclear whether or not said subject has a gene breakage in said gene of interest, sequencing or having sequenced at least a portion of a fused nucleic acid resulting from said gene breakage to identify the nucleic acid sequence fused to, or newly immediately adjacent to, said gene of interest. In alternative embodiments, the process (a) comprises performing an ISH (In Situ Hybridization). In alternative embodiments, said in situ hybridization analysis comprises a fluorescence in situ hybridization (FISH) analysis. In alternative embodiments, said fluorescence in situ hybridization analysis comprises hybridizing a plurality of differently labeled nucleic acid probes to said gene of interest, wherein said plurality of differently labeled nucleic acid probes yield signals of a distinct first and second color if said gene of interest has undergone a translocation, and a signal of a third color resulting from the combination of said first and second color if said gene of interest has not undergone a translocation.

In alternative embodiments of methods as provided herein, the sequencing or having sequenced comprises generating a cDNA from a transcript of said fused nucleic acid. In alternative embodiments, the sequencing or having sequenced comprises amplifying said cDNA and sequencing or having sequenced nucleic acids produced by said amplification. In alternative embodiments, the sequencing comprises performing a PCR, optionally a multiplex PCR (mPCR), to amplify the gene of interest fused to one or more potential fusion partners or portions of the gene of interest fused to portions of one or more potential fusion partners.

In alternative embodiments, provided are therapeutic methods comprising: assessing the translocation state of a gene of interest using a method as provided herein; and, if said gene of interest has undergone a translocation, administering a therapeutic agent or commencing with a therapy which is selected: based upon the finding of the identity of the nucleic acid fused to said gene of interest; or, based on characterization of the gene or genes involved in the gene breakage.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1 schematically illustrates an exemplary method as provided herein, which indicates that gene breakage analysis can be done by ISH (for example, FISH) or IHC (immunohistochemistry), and that sequencing of the breakage region is performed if the ISH or IHC is positive for a breakage or if it is unclear whether or not there is a breakage.

FIG. 2 and FIG. 3 are schematic illustrations of exemplary protocol steps and assay design for converting RNA to cDNA, including PCR probe design, and library preparation, as discussed in detail in Example 1, below.

FIG. 4 schematically illustrates an exemplary method for designing probes to be used in FISH protocols, as discussed in further detail, below.

FIG. 5 schematically illustrates an exemplary method for designing probes to be used in FISH protocols, and the results of the FISH, as discussed in further detail, below.

Like reference symbols in the various drawings indicate like elements.

### DETAILED DESCRIPTION

In alternative embodiments, provided are methods for identifying in a biological sample, such as a biopsy or cell or a tissue sample, a bone marrow smear, a cytological sample, a serum sample, a cytological blood sample, an aspirate sample, a liquid biopsy, a blood smear, a paraffin embedded tissue preparation, an enzymatically dissociated tissue sample, an uncultured bone marrow, an uncultured amniocyte and/or cytospin preparation, or a tissue culture preparation, or other biological sample, the presence of a genetic abnormality that leads to aberrant expression or function (for example, gain or loss of function, tissue specificity and/or downstream effects) of an RNA transcript or protein, and characterizing that genetic abnormality. Also provided are kits for practicing methods as provided herein. For a companion diagnostic or for precision medicine, the detection of the presence of the genetic abnormality as determined by methods as provided herein can be an important piece of information, for example, this information can be specifically important in devising strategies or therapies for patient care and monitoring, clinical research, identification of clinical biomarkers and/or for the development of companion diagnostics for therapeutics.

In alternative embodiments, methods as provided herein detect and characterize the breakage of a specific gene, for example, methods as provided herein can detect and characterize gene breakages including chromosomal translocations, gene rearrangements and/or gene inversions. For example, methods as provided herein can detect and characterize chromosomal translocations, gene rearrangements and/or gene inversions in genes where there are multiple potential partners for the translocation, rearrangement, or inversion event; for example, in the MLL (mixed lineage leukemia; see Meyer et al. 2013) gene at 11q23; and, in the ALK (anaplastic lymphoma kinase, also known as ALK tyrosine kinase receptor or CD246; see Du X. et al, 2018) gene, thus aiding in the diagnosis and treatment of mixed lineage leukemia (MLL) and leukemias with MLL translocations such as infant leukemia, therapy-related leukemia, acute myelogenous leukemia (AML), T-cell ALL, B lineage ALL, myelodysplastic syndrome (MDS), lymphoblastic lymphoma, and Burkitt's lymphoma; and, ALK fusion protein-related cancers, including anaplastic large-cell lymphomas, diffuse large B-cell lymphomas, systemic histiocytosis, inflammatory myofibroblastic tumors, esophageal squamous cell carcinomas and non-small-cell lung carcinomas.

In alternative embodiments, methods as provided herein comprise an approach or test comprising use of: first, an ISH (In Situ Hybridization, such as a Fluorescent ISH, or FISH) or an IHC (immunohistochemistry) or equivalent assay on DNA, RNA or nucleic acid complements thereof in a biological sample to detect a genomic abnormality such as a gene breakage, for example, a chromosomal translocation, gene rearrangement and/or gene inversion; and second, for those biological samples shown to be positive for a genetic abnormality or where it is unclear whether or not there is an abnormality, sequencing or polymerase chain reaction (PCR) of the genetic abnormality region, for example, the gene breakage region, to specifically characterize which gene or genes are involved in the gene breakage.

In alternative embodiments, in the first step, the ISH or IHC or equivalent can be a standardized ISH or IHC assay protocol, and can provide a standardization for entry into the second step sequencing assay. The sequencing or PCR assay protocol also can be standardized.

In practicing methods as provided herein, because the biological samples (i.e., the patients) are first screened out by the ISH or IHC assay before the sequencing, the overall test costs per patient is less expensive than when only a full sequencing approach is applied to all patients. Another potential benefit of methods as provided herein is that the results of the ISH or IHC or equivalent test would be available faster than a sequencing only approach, and ISH and IHC are widely and commonly used in laboratories across the world.

Additional potential benefits of methods as provided herein are that the patients or samples that are tested and found to have breaks (translocations, rearrangements, inversions) or where it is unclear whether or not there is a break can have the full suite of information from sequencing, such as the gene partner, where the break occurs, and what type of break.

In alternative embodiments, approaches of methods as provided herein can also take advantage of each modalities' fit-for-purpose. The ISH or IHC or equivalent assay can be used for an initial screen followed by sequencing for deeper information on what is the nature of the break. This also avoids the need for an equivocal zone of interpretation for the test or for a sample to be labeled as equivocal.

In addition to samples where ISH or IHC or equivalent assay are clearly positive for a gene breakage, a sample that does not meet the lower threshold for being negative, but positivity is questioned, are also sent for sequencing. The diagnostic cut-off of the ISH or IHC assay will depend on correlation to clinical outcome, and can range from greater than 0% to 1% to up to less than 100% (e.g., about 80% or 90%, or any subrange in this broader range) of cells of interest showing a fused signal or expressing the (for example, fused or truncated) protein of interest—in which case the cells are sent for sequencing. Specifically, in the case of ALK FISH, a cut-off of greater than about 50% cells containing a fused signal is considered diagnostically positive, in other words, if greater than about 50% cells are diagnostically positive, then the cells are sent to screening. In alternative embodiments, if a ISH or IHC or equivalent assay finds that greater than about 1%, 5%, 10%, 20%, 30%, 40%, 50%, 60%, 70% or 80% of the cells in a sample are diagnostically positive for a gene breakage, then that sample is considered sufficiently positive to be sent for sequencing.

In alternative embodiments, other advantages of the methods as provided herein are:
The methods as provided herein combines a lower cost option (ISH or IHC) with a higher cost option (sequencing). The high cost test would only be applied in cases where there is a reasonable expectation of there being a break in the gene of interest in contrast to a high cost only option where all samples are sequenced.
In the first step of methods as provided herein, the ISH or IHC would have a faster turn-around-time than a sequencing only approach.
Use of the methods as provided herein can drive higher pathology lab adoption because more labs can perform the ISH or IHC as part of their normal workflow (worldwide). ISH or IHC testing could be decentralized. Because sequencing is run in less labs, having a smaller concentration of samples that need to be sequenced could allow for centralization of the sequencing.
Information from sequencing is only captured when needed.
If ISH or IHC is used, only one ISH or IHC probe set is needed.
Approaches as provided herein can take advantage of each modalities fit for purpose. This would be screening for potential breakage for ISH or IHC and deeper data collection regarding a breakage for sequencing in specific instances (ISH or IHC positive samples).
The amount of human sample is typically limited in size and number of cells. Also, there can be quality issues with the tissue. Exemplary approaches as provided herein can allow for the tissue to be used in a way that more information (including tissue context, expression data, and genetic/genomic data) is captured from the same sample providing the ability to do quality control on the block for all of the data captured and data correlation between the two methods.

In alternative embodiments, methods as provided use a combination of ISH or IHC and nucleic acid sequencing of the suspected or known gene breakage region (for example, sequencing-based fusion detection)—which previously have not been linked, in contrast to simply sequencing the suspected region of gene breakage. In alternative embodiments, all parts of the methods as provided herein are performed with automation, and without automation for each step or modality, the turn-around-time, hands on time, and cost could be prohibitive. Additionally, in some embodiments, methods as provided herein for the first time are entirely automated, and the ISH and the sequencing are integrated as part of a complete workflow. Additionally, the methods as provided herein for the first time provide an algorithm that links the two approaches.

### In Situ Hybridization (ISH) or Immunohistochemical (IHC) Assays

In alternative embodiments of the methods as provided herein, in the first step an ISH or IHC is performed on a biological sample, for example, a biopsy or a cell or tissue sample, which can be a cytological sample, blood sample, an aspirate sample, a liquid biopsy, a blood smear, a paraffin embedded tissue preparation, an enzymatically dissociated tissue sample, an uncultured bone marrow, an uncultured amniocyte and/or cytospin preparation, or a tissue culture preparation. The first portion of the process detects whether a breakage has occurred, and in alternative embodiments an in situ hybridization (ISH) or an immunohistochemical (IHC) assay is performed to determine if a breakage is present. The ISH or IHC can be an automated ISH or IHC. See Example 1 for an exemplary automated FISH protocol. It will be appreciated that any ISH or IHC protocol which can indicate the presence of a breakage may be utilized in the methods provided herein.

In alternative embodiments, a break-apart or split-signal ISH assay can be performed on the gene or a transcript of interest. However, only that the gene or transcript is broken, and possibly one partner of this re-joining event is fused to the gene of interest, can be detected by this approach. The identity of the partner in the breakage and the molecular characterization of the break cannot be determined if there is more than one partner, or break type, with a single probe set. This can be a disadvantage for a split-signal (F)ISH. Alternatively, multiple probes or probe sets can be used if there is more than one partner, or break type; one probe set is used for each partner.

If the ISH or IHC assay is positive for a break or if it is unclear whether or not there is a break, the sample would then be sequenced to confirm the ISH or IHC result and what the nature of the break is. For example, the type of break and the re-joining partner are determined by sequencing. If the ISH or IHC assay is negative for a break, the testing would end.

In alternative embodiments, in situ hybridization (ISH) or IHC is performed, for example, on slides from formalin-fixed, paraffin embedded (FFPE) tissue or cell blocks, where the tissue or cells can be taken from biopsies. In alternative embodiments, the slides are prepared by methods comprising use of an automated instrument which takes the slides through a series of pre-treatment steps, incubates the slides with the probes to the gene of interest, and washes the slides. The slides can then be prepared for microscopy, visualization, and analysis by dehydration, mounting and cover slipping.

In alternative embodiments, the slides are visualized and analyzed for signal. For example, in one embodiment, fluorescently tagged probes are used, where each probe of a pair of probes has a different emission color or frequency, for example red and green. The pair of probes is designed to hybridize to a gene of interest, for example, a protein coding sequence, or exon, or a gene of interest. If two separate signals (for example red and green) are present (detected) in tumor cells subject to the ISH, then the gene of interest is broken, for example, the gene is translocated, rearranged or inverted. If a yellow signal is observed, the probes are spatially close enough to be interpreted as the gene of interest being physically intact and free from detectable translocations, rearrangements, and inversions.

If the number of cells with a positive signal (red and green separate signals) in a tissue sample or cell sample reaches a defined threshold, then the nucleic acid sample is sequenced to determine (identify, characterize) the partner gene that has rejoined following the breakage event. If it is undetermined if that threshold is met, the sample is also sequenced. If it is determined if that threshold is not met, the sample is not sequenced.

In alternative embodiments, the sample is prepared for sequencing by extracting the nucleic acid (for example, RNA) and then converting the RNA to cDNA, followed by an adaptor ligation. This cDNA is used for sequencing (for example, Next Generation Sequencing (NGS)) library preparation. The NGS library preparation can be performed by a multiplex PCR step with primers designed to amplify fusions between a gene of interest and one or more potential fusion partners. The primers may be designed to allow for a subsequent universal PCR.

Methods as provided herein can be generalized and can be all automated, or can be applied to performing the automated steps manually. Probes used in methods as provided herein can be designed to any number of genes where breaks in the gene need to be identified (for example translocations, rearrangements, and inversions) and any number of potential fusion partners with those genes. Also, the probes described here can labeled fluorescently, or using an equivalent detectable label or moiety, for example, where they can be individually visualized, for example, as red and green signals or dots, where the spatial location in an intact gene brings the fluorescent or detectable signal close enough (i.e. the physical location on the chromosome) to be visualized/analyzed as a hybrid unique, for example, as a yellow signal or dot (where the physical proximity of the two different detectable labels or moieties result in an emission, color or signal different from that emitted or generated by either of the two probes when not in proximity to each other; for example, the red and green probes when in physical proximity generate a yellow color). However, other color or detectable emission combinations are possible and different probes can be labeled with different color or detectable label signals. Thus, pairs of probes used to practice methods as provided herein can comprise any of multiple combinations of probes with multiple combinations of fluorophores or detectable moieties. For example, fluorescent dyes (fluorophores) suitable for use as labels in methods as provided herein can be selected from any of the many dyes suitable for use in imaging applications, for example, flow cytometry, and a large number of dyes are commercially available from a variety of sources, such as, for example, Molecular Probes (Eugene, Oreg.) and Exciton (Dayton, Ohio), that provide great flexibility in selecting a set of dyes having the desired spectral properties. Examples of fluorophores or labels suitable for use as labels in methods as provided herein include, but are not limited to: 7-amino-4-methylcoumarin-3-acetic acid (AMCA), TEXAS RED (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate; 4-acetamido-4′-isothiocyanatostilbene-2,2′disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (TEXAS RED™), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (for example, Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue acetylazide (Molecular Probes, Inc.), Cascade Yellow; Quantum Dot dyes (Quantum Dot Corporation); Dylight™ dyes from Pierce (Rockford, Ill.), including Dylight 800™, Dylight 680™, Dylight 649™, Dylight 633™, Dylight 549™, Dylight 488™, Dylight 405™; or combinations thereof. Quantum dots may also be employed.

A FISH sample can be read using a variety of different techniques, such as, for example, by microscopy, flow cytometry, fluorimetry, etc. Microscopy, such as, for example light microscopy, fluorescent microscopy or confocal microscopy (such as confocal laser scanning microscopy (CLSM) or laser confocal scanning microscopy (LCSM), or spinning disk confocal microscopy), or programmable array microscopes (PAM), can be used for detecting light signal(s) from a FISH sample. In embodiments in which oligonucleotides are labeled with a fluorescent moiety, reading of the contacted sample to detect hybridization of labeled amplification products can be carried out by fluorescence microscopy.

Also, methods as provided herein can comprise additional steps applied to convert the fluorescent signal to a chromogenic signal, and this can facilitate the visualization and analysis by conventional, brightfield microscopy. In addition to the analysis of slides by a human reader, dots or signals may also be analyzed by automated imaging and/or image analysis. There can be additional image processing and interpretation performed to add additional information such as how large the spots are, are there spots overlapping, and other features.

Any FISH protocol can be used to practice methods as provided herein, including methods as described for example, in U.S. Pat. No. 8,034,917; or a multicolor FISH-based method, for example, a FISH that allows the visualization of all 24 autosomes, each in a different color, for example, a “chromosome painting” approach as described in Speicher et al, Nature Reviews (2005) 6:782-792; Liehr et al, Histol. Histopathol. (2004) 19:229-37; Matthew et al, Methods Mol. Biol. (2003) 220: 213-33. A FISH protocol that can be used to practice methods as provided herein can also include: a multiplex-FISH protocol, for example, as described in Speicher et al., Nature Genet. (1996) 12: 368-375; or, a spectral karyotyping as described by Schrock et al., Science (1996) 273: 494-497; or, a combined binary ratio labeling (COBRA) as described by Tanke et al., Eur. J. Hum. Genet. (1999) 7: 2-11. Any method that can identify intrachromosomal rearrangements can be used, including a method used to identify intrachromosomal rearrangements on genomic samples from non-dividing or metaphase cells.

In alternative embodiments, oligonucleotide primers or probes are designed to target and amplify segments of genes or transcripts of interest. For example, primer or probe can be designed to be as close as possible to the edge of a suspected fusion exon. Primers or probes can be nested or non-nested primers in relation to the exons or protein coding sequences for which they are designed to amplify. Primers or probes can be designed, for example, as described in U.S. Pat. No. 8,034,917. Primers or probes comprise oligonucleotides that have a nucleotide sequence that is complementary to a region of a target nucleic acid. A primer or probe binds to the complementary region and is extended using the target nucleic acid as the template under primer extension conditions. A primer can be in the range of about 15 to about 60 nucleotides or longer. A primer can include moieties that comprise the known purine and pyrimidine bases, and also other heterocyclic bases that have been modified, for example, can include methylated purines or pyrimidines, acylated purines or pyrimidines, alkylated riboses or other heterocycles.

In alternative embodiments, in silico methods are used to design oligonucleotide primers or probes used to practice methods as provided herein. In alternative embodiments, oligonucleotide primers or probes used to practice methods as provided herein are designed for maximum specificity and high resolution for gene breakage detection, for example, in silico methods can be coupled with high fidelity oligo synthesis to produce primers or probes (for example, SureFISH™ probes, Agilent Technologies, Santa Clara, Calif.) to have maximum specificity and higher resolution than traditional FISH probes. Probes can be specifically designed for translocation detection.

In alternative embodiments, primers or probes used to practice methods as provided herein can comprise unique long oligonucleotides that are tiled across the targeted chromosomal region avoiding non-unique portions of the genome. Using knowledge of translocation breakpoints, probes can be designed to detect the translocated sequences using both break-apart and dual fusion strategies. An in silico design methodology and de novo synthesis of probes enables optimization of design characteristics such that each probe provides balanced signals, facilitating the detection of chromosomal rearrangements, and that can target almost any genomic region.

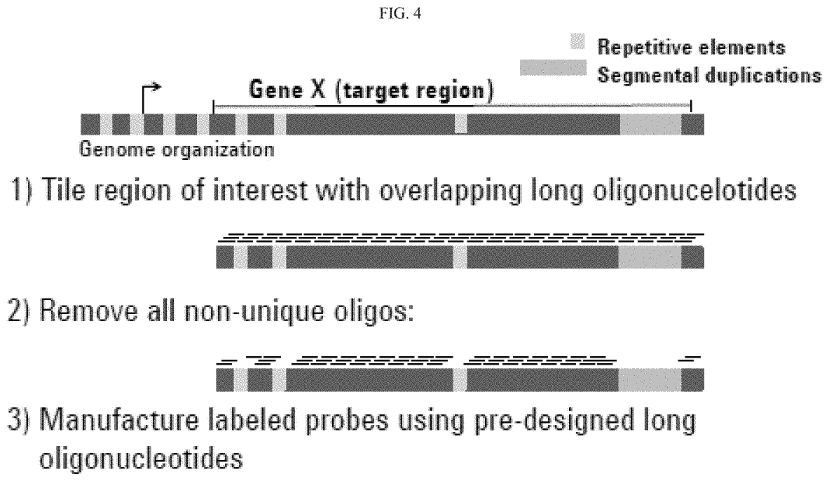

For example, in alternative embodiments, probes are designed by first tiling a region of interest with overlapping long oligonucleotides; remove all non-unique oligos (lack of repetitive sequences in the selected probes results in specific signal and low background); and then labeling the probes, for example, as illustrated in FIG. 4.

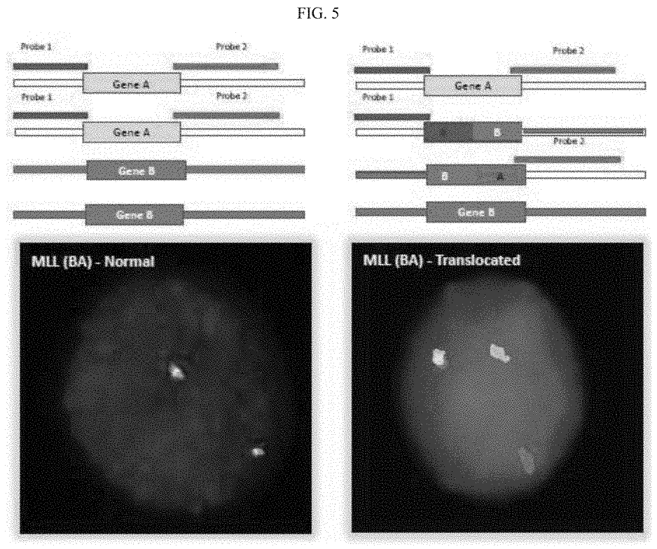

Only oligos that are contained within and unique to the targeted region are used in the probe, resulting in specific signals and eliminating the need for suppressive hybridization reagents such as Cot1. The use of break-apart probes for the detection of translocations using FISH is illustrated in FIG. 5, where each member of the pair of probes has a different label (in this example, red and green), and when the probes remain in proximity to each other (no gene breakage) a third emission is detected (in this example, the color yellow), but when the probe pairs are separated because of a gene breakage, only the original label emissions remain (in this example, red and green). FIG. 5 illustrated the FISH detection of an MLL gene translocation.

In alternative embodiments, either fusion-signal FISH or split-signal FISH are used, for example, as described in Burg et al, Leukemia (2004) 18, 895-908. Fusion-signal FISH uses two probes located in the two genes, which are involved in the chromosome translocation. In normal situations, green and red signals will be present, in other words, the two genes are sufficiently separated that the green and red signals do not interact to create a third signal (such as a yellow signal). In case of a translocation, a green and a red signal colocalize and generate a yellow signal (in other words, the two genes are sufficiently close that the green and red signals do interact to create a third signal (such as a yellow signal)), together with the separate green and red signals of the unaffected genes.

Split-signal FISH uses two probes positioned at opposite sides of the breakpoint region in one of genes, which are involved in the chromosome translocation. In normal situations, two yellow signals will be present, while in case of a translocation separate green and red signals will be present together with the colocalized signal of the unaffected gene.

### Immunohistochemistry (IHC)

In alternative embodiments of the methods as provided herein, an IHC (immunohistochemistry) assay is used to identify and characterize a gene breakage in a biological sample. In this embodiment, antibodies that can specifically identify a gene translocation, gene rearrangement and/or gene inversion event are used. The antibodies can be directly or indirectly labeled with a detectable moiety for IHC identification and detection. The antibody or antibodies can specifically bind to an epitope unique to a protein fusion event, for example, a fusion event caused by a gene translocation. Any IHC assay or protocol known in the art can be used to practice methods as provided herein, see for example, Hansen et al, Appl. Immunohistochem. Mol. Morphol. 2006 March; 14(1):115-21, which describes IHC protocols and the optimization of antibodies for use in IHC; or Perren et al, Am. J. Pathol. 1999 October; 155(4):1253-60, which described use of IHC to identify PTEN mutations; or, Argani, et al, American J. Surgical Path.: February 2005, Vol 29(2), pg. 230-240, which describes identifying an alpha-TFEB gene fusion using IHC.

Also for example, Luk et al, Arch Pathol Lab Med, Vol 142, August 2018, describe use of IHC as an effective assay for identifying ALK and ROS1 gene rearrangements in non-small lunch cancer cells. They described several antibodies that are available for the detection of ALK expression in lung adenocarcinomas, including 5A4 (Novocastra NCL-ALK, Leica, Wetzlar, Germany), D5F3 (Ventana, Tucson, Ariz.), ALK1 (Dako, Carpinteria, Calif.), and 1A4 (Origene, Rockville, Md.); and, the D4D6 clone of ROS1 antibody (Cell Signaling Technology, Danvers, Mass.) to detect ROS1 gene rearrangements.

### Nucleic Acid Sequencing

In alternative embodiments of the methods as provided herein, sequencing, such as Next Generation Sequencing, is performed on a nucleic acid sequence identified as having a breakage, or on a nucleic acid sequence if it is unclear whether or not there is a breakage in that nucleic acid sequence. Thus, sequencing is performed only on samples not definitely identified as negative for a gene breakage.

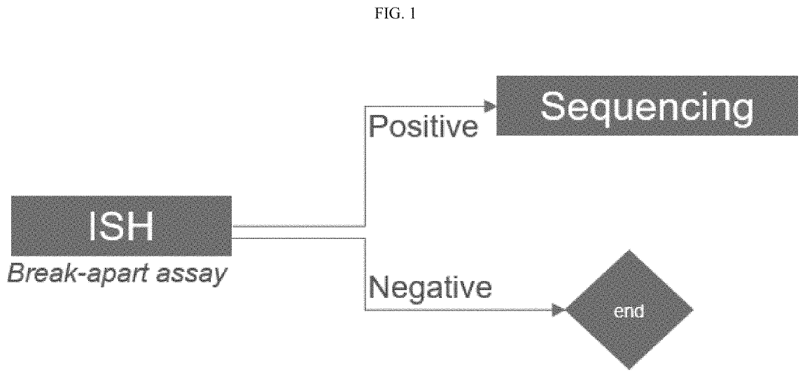

In alternative embodiments, sequencing is performed to characterize and identify the gene or transcript of interest and a fusion partner, for example, in the case of a translocation, for example, sequencing can identity the (fusion) partner gene, as illustrated in FIG. 1.

As discussed above, a sample can be prepared for sequencing by converting the RNA to cDNA, where the cDNA is used for sequencing (for example, Next Generation Sequencing (NGS)) library preparation. Preparation of the cDNA can be followed by an adaptor ligation, and use of a PCR step with primers that allow for a subsequent universal PCR. Amplifying a cDNA library with universal primers allows for hybridization-based capture methods. In alternative embodiments, the PCR utilizes primers which amplify fusions between a gene of interest and any number of other sequences which may potentially be fused to the gene of interest. Primers can be designed as described herein and can be used to amplify both a gene of interest (for example, MLL) and its fusion partner or partners or portions thereof.

In alternative embodiments, multiplex polymerase chain reaction (mPCR), a variant of PCR, is used. By using mPCR two or more target sequences (for example, a particular gene of interest fused to any one of two or more potential fusion partners) can be amplified by including more than one pair of nucleic acid amplification, or PCR, primers in a single nucleic acid amplification reaction. In alternative embodiments of the mPCR, two or more target sequences are amplified in parallel, for example, as described in Del Favero et al, WO/2018/108421. For example, a first amplification reaction can use target specific primers that amplify the target nucleic acid molecules, and the target specific primers can also comprise universal tags. The universal tags are incorporated into the amplification products as the reaction proceeds. In the second amplification reaction, universal primers designed to hybridize with these tag sequences are then used to amplify the amplification products from the first amplification. This second amplification involves primers that incorporate any further sequences needed for further processing and identification purposes, such as adaptors. The “universal” amplification is performed independently of the specific target sequence of the initial target molecule that is amplified, and it relies upon the incorporation into the amplification products from the first amplification reaction of additional sequence (so-called “universal tags”) that can act as primer binding sites in a second amplification. The primer region of the primers in the second amplification corresponds to the universal tag sequence, and primers including such primer regions are “universal primers”.

In alternative embodiments, mPCR also can be carried out as described in Goossens et al (2009) Human Mutation O, 1-6, who show mPCR to be a front-end method for massive parallel sequencing and its application to simultaneous variation and copy number variation (CNV) detection.

Following this universal PCR, the sample is then sequenced, for example, on a sequencing platform, using for example, high throughput sequencing such as Next Generation Sequencing (NGS) or massively parallel signature sequencing (or MPSS). In alternative embodiments, high-throughput sequencing can be used on platforms as designed by, for example, Illumina, Qiagen or ThermoFisher Scientific; or as described for example, in U.S. Pat. Nos. 9,738,930; or 9,080,210; or U.S. patent application nos. US20190055597 A1; or US20180363047 A1.

The primary sequencing data can be exported to a sequence data analysis platform or program. This analyzed data can then be used to create a report summarizing the analyzed data.

### Biological Samples

In alternative embodiments, methods as provided herein comprise analysis of genes or transcripts from individuals suspected of having cancer, and the analysis is carried out on biological samples taken from these individuals. These biological samples can comprise a biopsy or a cell or tissue sample, a cytological sample, or can be in the form of bone marrow smears (for example bone marrow aspirate smears), a serum sample, a cytological blood samples, an aspirate sample, a liquid biopsy, a blood smear, paraffin embedded tissue preparations (for example, as described by Canene-Adams, Methods Enzymol. 2013; 533:225-33), enzymatically dissociated tissue samples, uncultured bone marrow, uncultured amniocytes and/or cytospin preparations, tissue culture preparations, and the like.

### Kits

In alternative embodiments, provided are kits for practicing methods as provided herein. A kit can comprise a subject oligonucleotide composition, or a plurality of pairs of PCR primers, where each pair of PCR primers amplifies a fusion between a gene of interest and a specific fusion partner of interest. The kit can comprise a polymerase, reagents for PCR (for example, a buffer, nucleotides, etc.), materials for fluorescent labeling of polymerase products, and/or a reference sample. The kit may also comprise reagents for performing in situ hybridization or immunohistochemistry analysis to determine if one or more particular genes of interest have undergone a breakage. The various components of the kit may be in separate vessels.

In addition to above-mentioned components, a kit can also comprise instructions for using the components of the kit to practice methods as provided herein. The instructions for practicing the subject methods can be recorded on a suitable recording medium. For example, the instructions can be printed on a substrate, such as paper or plastic, etc. The instructions can be present in the kits as a package insert, in the labeling of the container of the kit or components thereof (for example, associated with the packaging or sub-packaging) and the like. In other embodiments, the instructions can be present as an electronic storage data file present on a suitable computer readable storage medium, for example CD-ROM, diskette, etc. In yet other embodiments, the actual instructions are not present in the kit, but ways to obtain the instructions from a remote source, for example via the internet, are provided. An example of this embodiment is a kit that includes a web address where the instructions can be viewed and/or from which the instructions can be downloaded, wherein optionally the web address or download can provide instructions for carrying out: tissue preparation; an ISH, a FISH or an IHC assay; PCR; cDNA production and/or for sequencing or preparation for sequencing, for example for NGS (Next Generation Sequencing).

Any of the above aspects and embodiments can be combined with any other aspect or embodiment as disclosed here in the Summary and/or Detailed Description sections.

As used in this specification and the appended claims, the singular forms “a,” “an” and “the” include plural referents unless the context clearly dictates otherwise. Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Automated FISH

This example demonstrates an exemplary protocol for automated FISH used to practice methods as provided herein.

### Exemplary Protocol for Automated FISH

FFPE tissue or cell blocks are sectioned to a thickness of 3 to 6 microns, and mounted on glass slides.
Slides are placed in an oven for drying. For example, they may be held at 60 deg C. for 60 minutes.
The slides are then held at 2 to 8 deg C. or used as soon as cooled to room temperature.
The probe or a cocktail of probes are brought to the working concentration and mixed (either through an automated mixer or manually) and then loaded onto the instrument.
The necessary reagents are loaded onto an automated platform (for example, a Dako OMNIS™ automated platform, Agilent).
The automated platform brings the slides through the following series of steps:
   Dewax and pre-treatment
   Target retrieval
   Rinses (water and alcohol)
   ISH pepsin
   A wash step
   Drying
   Probe or probes application
   Denaturation step
   Hybridization step
   Stringent wash(s)
   Dry
The slides are dehydrated through a series of alcohol steps.
The slides are dried.
Mounting media is applied to the slides.
A cover slip is placed on the slide.

### Exemplary Visualization, Analysis, and Algorithm Protocol

The slides are visualized under a microscope with florescence viewing capabilities.
The number of tumor cells with two separate color dots (for example red and green) is determined and the number of tumor cells with a single, different color dot (for example yellow) is determined. Note: dots may also be referred to as signal.
If the number of tumor cells with red and green dots reaches a certain threshold, the sample is considered positive and then sequencing is performed.
If the number of tumor cells with red and green dots does not reach a certain threshold (or if there are no tumor cells with red and green dots present), sequencing is not performed, and the sample is considered negative.
If the number of tumor cells with red and green dots is not able to be determined or if an exception is made for the sample, then the sample is sequenced.

### Exemplary Protocol for: Isolation of Sample RNA, Reverse Transcription to cDNA, NGS Library Preparation

RNA is isolated from FFPE samples through standard methods of extraction.
A quality control step is performed.
cDNA is prepared and ligated to an adaptor
Multiplex PCR is performed with target-specific primers containing sequences complementary to universal PCR primers to amplify a gene of interest fused to one or more potential fusion partners
Universal PCR is performed with universal PCR primers

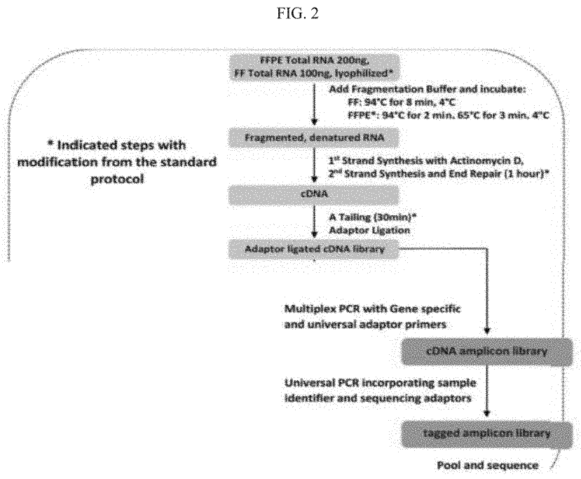

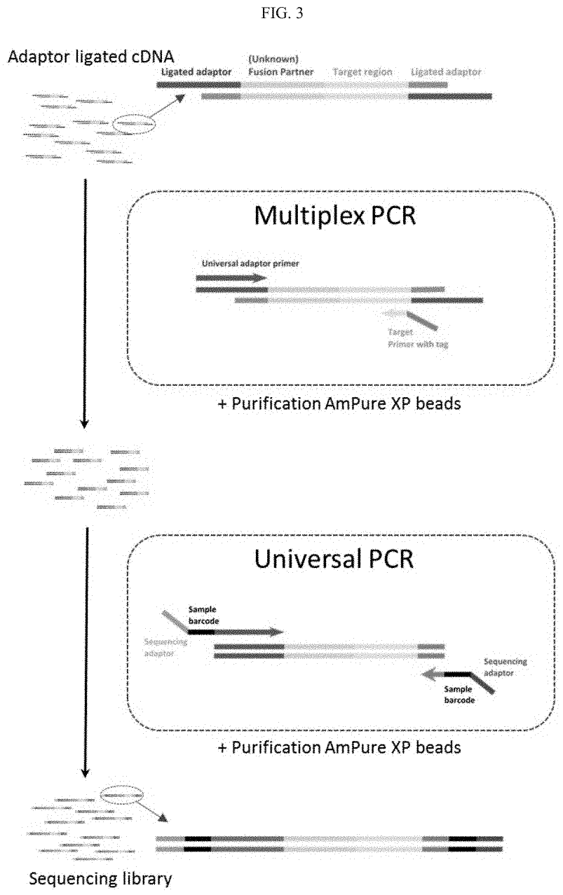

See FIG. 2 and FIG. 3 for schematic illustrations of exemplary protocol steps for converting RNA to cDNA, and assay and probe design, and library preparation.

In alternative embodiments, nested or non-nested PCR is used. In alternative embodiments, the fusion primer (i.e., the primer located in the gene interrogated for fusion events) is designed to be as close as possible to the edge of the fusion exon. In case of nested PCR the initial fusion detection primer is by design located further from the exon boundary, which can lead to reduced sensitivity; this is not the case for the non-nested PCR approach. Given that nested PCR increases specificity, non-nested PCR might have have reduced specificity. Additionally workflow may be shorter for the non-nested PCR method.

In alternative embodiments, advantages of using an RNA Direct protocol such as that illustrated in FIG. 2 and FIG. 3, are:
cDNA Library can be easily stored because DNA is more stable than RNA and hence can preserve precious samples for other and/or later use.
Library can be used for whole transcriptome analysis in cases where a targeted protocol such as the MASTR Reporter™ software platform (Agilent) protocol is not yielding result based on FISH screening.
Library can be used with capture-based methods (for example, SURESELECT™ (Agilent Technologies, Santa Clara, Calif.) Target Enrichment System for a Paired-End Multiplexed Sequencing Library),
Data analysis is directed to fusion detection.

### Example 2: Sequencing and Data Analysis

This example demonstrates an exemplary protocol for sequencing and data analysis used to practice methods as provided herein.

### Sequencing and Data Analysis Summary

The sequencing library is loaded onto a sequencing platform.
The primary data is obtained and transferred to the data analysis platform.
Quality control steps are performed.
A final report with a summary of the presence of a gene break and what the nature of the gene break is, including the partner gene that is rejoining the gene-of-interest (if applicable) can be generated.

### Example 3: Exemplary Approaches to Gene Breaks

This example demonstrates an exemplary method or protocol as provided herein for detecting and characterizing gene breaks.

First an ISH step is performed. Tissue or cell Formalin Fixed Paraffin (FFPE) embedded blocks are sectioned at a thickness of 3 to 6 micron and placed on microscope slides. These slides are then dried in an approximate 60 deg C. oven for approximately 60 minutes. The slides are then cooled to room temperature and used directly after the cool down to room temperature or stored at 2-8 deg C. until use. If stored at 2 to 8 deg C. they are equilibrated to room temperature before use. Before staining commences, the probe or probe cocktail is prepared by bringing the probe(s) to the appropriate staining concentration, also called the working concentration (which may be empirically determined for each set of probes for each gene), and mixed via an automated mixer. The probes and other reagents needed for staining are loaded onto the automated platform (for this description the exemplary Dako OMNIS™). The slides are loaded onto the instrument and the procedure is started. The slides are dewaxed and exposed to pre-treatment steps. The slides are then brought through a series of water and alcohol rinses. The slides are treated with an ISH pepsin (protease) digestion step followed by another wash step. The slides are dried and then the probe or probe cocktail is applied. Following the probe application, there is a denaturation step then a hybridization step. The slides then go through stringent wash or washes. The slides are dried then it is recommended they are taken through a series of alcohol steps, and which can be dried again. Mounting media is applied, and then a coverslip is applied. Slides are held in the dark until they are visualized and/or analyzed.

Slides are visualized under fluorescence microscopy. First the slides are viewed under low power for a tissue overview, then viewed under increasing magnification as needed (for example, medium power) to determine the presence and location of the tumor cells. The slides are then viewed under high power for the dot or signal identification and interpretation. The number of cells that have both red and green signals are determined. This is done for tumor cells in the tissue or cell sample. The number of tumor cells with a yellow signal is also determined. If the number of cells with both red and green signals, or the percentage of total tumor cells, reaches a threshold, which can be empirically or logically determined for each gene of interest, then the samples are considered positive and sequenced. If the number of red and green signals does not reach this threshold, then the sample is considered negative and not sequenced. Additional exceptions may be determined for each gene of interest or generally for gene breaks. If a sample falls into one of these exception categories, it may also be sequenced. If the number of positive cells (with both red and green signals) and negative cells (yellow signals) cannot be determined, the sample will also be sent to sequencing.

When a sample has been identified for sequencing, the total RNA is isolated through a standard method of extraction. The RNA is then fragmented and denatured. 1^{st} and 2^{nd} stgrand RNS synthesis (reverse transcription) is performed to produce the resultant cDNA. Adaptors are then ligated to the cDNA. This pool of cDNA then goes through a multiplex PCR step with gene specific and universal adaptor primers to create the cDNA amplicon library. Following a purification step, a universal PCR step is performed with a sample identifier and sequencing adaptors. A purification step is performed to produce a sequencing library.

This sequencing library is then used with a sequencing platform to obtain primary sequencing data. Data analysis can then be summarized in a report with the information such as if the gene of interest has a break (confirmation of the ISH), what the nature of the break is, and other information for diagnosis or precision medicine. For example, this other information could include the ISH results or other tests done on the sample.

A number of embodiments of the invention have been described. Nevertheless, it can be understood that various modifications may be made without departing from the spirit and scope of the invention. Accordingly, other embodiments are within the scope of the following claims.

What is claimed is:

1. A method for identifying and characterizing a gene breakage in a biological sample, wherein the gene breakage is characterized or identified by a nucleic acid sequencing only after the biological sample is positively identified as having a gene breakage by an ISH (in situ hybridization) or an IHC (immunohistochemistry) assay, or the gene breakage is characterized or identified by a nucleic acid sequencing only if the biological sample is not positively identified as not having a gene breakage, the method comprising:
(a) providing or having provided a biological sample;
(b) providing or having provided probes designed to detect the gene breakage in the gene or its corresponding transcript;
(c) detecting the presence or absence of a gene breakage in the biological sample using the ISH (in situ hybridization) or the IHC (immunohistochemistry) assay;
(d) sequencing or having sequenced nucleic acid from the biological sample if a gene breakage is detected in the biological sample to characterize the gene or genes involved in the gene breakage, or sequencing or having sequenced nucleic acid from the biological sample if it is not positively determined that a gene breakage has not occurred in the biological sample; and if it is positively determined that no gene breakage has occurred (or is present) in the biological sample the sample is not sequenced or had sequenced; and
(e) analyzing or having analyzed the sequence of the sequenced nucleic acid to identify or characterize the gene or genes involved in the gene breakage.

2. The method of claim 1, wherein the gene breakage comprises a gene translocation, a gene rearrangement or a gene inversion.

3. The method of claim 1, wherein the ISH is a Fluorescent ISH, or FISH assay.

4. The method of claim 1, wherein the biological sample is or comprises a biopsy or a cell or tissue sample, or can be in the form of a bone marrow smear, a cytological sample, a serum sample, a cytological blood sample, an aspirate sample, a liquid biopsy a, blood smear, a paraffin embedded tissue preparation, an enzymatically dissociated tissue sample, an uncultured bone marrow, an uncultured amniocyte and/or cytospin preparation, or a tissue culture preparation.

5. The method of claim 1, wherein the detecting the presence or absence of a gene breakage in the biological sample comprises: counting the total number or percentage of cells having signals indicating the presence of a gene breakage, and if the total number or percentage reaches a threshold, which can be empirically or logically determined for each gene of interest, then the samples are considered positive and sequenced.

6. The method of claim 1, wherein the detecting the presence or absence of a gene breakage in the biological sample comprises: counting the total number or percentage of cells having signals indicating no gene breakage, and if the total number or percentage of gene breakage negative cells does not reach a threshold that positively indicates no gene breakage in the sample, which can be empirically or logically determined for each gene of interest, then the samples are considered positive and sequenced (sequencing is performed only on samples not definitely identified as negative for a gene breakage).

7. The method of claim 1, wherein the sequencing of the nucleic acid from the biological sample comprises use of a high throughput or Next Generation Sequencing or massively parallel signature sequencing (or MPSS).

8. The method of claim 7, wherein the sequencing comprises first converting cell transcripts or mRNA to cDNA, where the cDNA is used for sequencing library preparation.

9. The method of claim 8, wherein the sequencing library preparation of the cDNA is followed by an adaptor ligation.

10. The method of claim 9, wherein the sequencing library preparation of the cDNA is followed by an adaptor ligation that comprises use of a multiplex PCR (polymerase chain reaction) (mPCR) step with primers that allow for a subsequent universal PCR step.

11. The method of claim 1, wherein the gene breakage is in an MLL (mixed lineage leukemia) gene or an ALK (anaplastic lymphoma kinase) gene.

12. The method of claim 1, wherein the probes are designed as fusion-signal FISH or split-signal (also called “break-apart”) FISH probes.

13. A method for diagnosing a disease or condition in an individual comprising use of the method of claim 1, for identifying and characterizing a gene breakage in a biological sample, wherein a specific gene breakage is associated with a particular disease or condition, and if the specific gene breakage is found to be present in a biological sample from the individual, then the individual can be diagnosed with that disease or condition.

14. A method for treating, ameliorating or preventing a disease or condition in an individual in need thereof comprising use of the method of claim 1 for identifying and characterizing a gene breakage in a biological sample, wherein a specific gene breakage is associated with a particular disease or condition, and if the specific gene breakage is found to be present in a biological sample from the individual, then the individual can be diagnosed with that disease or condition and subsequently treated for that disease or condition.

15. The method of claim 14, wherein the breakage, or specific gene breakage, is in an:
(a) MLL (mixed lineage leukemia) gene, and the disease or condition diagnosed and treated is: an infant leukemia, a therapy-related leukemia, an acute myelogenous leukemia (AML), a T-cell ALL, a B lineage acute lymphoblastic leukemia (ALL), a myelodysplastic syndrome (MDS), a lymphoblastic lymphoma or Burkitt's lymphoma, or
(b) ALK (anaplastic lymphoma kinase gene), and the disease or condition diagnosed and treated is: an anaplastic large-cell lymphoma, a diffuse large B-cell lymphoma, a systemic histiocytosis, an inflammatory myofibroblastic tumor, an esophageal squamous cell carcinoma or a non-small-cell lung carcinoma.

16. A method for diagnosing a disease or condition in an individual using a method of claim 1, wherein a specific gene breakage is associated with a particular disease or condition, and if the specific gene breakage is found to be present in a biological sample from the individual, then the individual can be diagnosed with that disease or condition.

17. The method of claim 16, wherein the breakage, or specific gene breakage, is in an:
(a) MLL (mixed lineage leukemia) gene, and the disease or condition diagnosed: an infant leukemia, a therapy-related leukemia, an acute myelogenous leukemia (AML), a T-cell ALL, a B lineage acute lymphoblastic leukemia (ALL), a myelodysplastic syndrome (MDS), a lymphoblastic lymphoma or Burkitt's lymphoma, or
(b) ALK (anaplastic lymphoma kinase gene), and the disease or condition diagnosed: an anaplastic large-cell lymphoma, a diffuse large B-cell lymphoma, a systemic histiocytosis, an inflammatory myofibroblastic tumor, an esophageal squamous cell carcinoma or a non-small-cell lung carcinoma.

18. A kit comprising components or materials for use in practicing a method of claim 1.

19. The kit of claim 18, wherein the components or materials for use in practicing a method of claim 1 comprise: PCR (polymerase chain reaction) reagents and/or probes.

20. The kit of claim 19, wherein the PCR is multiplex PCR (mPCR);
probes and/or reagents for conducting ISH (in situ hybridization) or an IHC (immuno-histochemistry).

21. The kit of claim 20, wherein the ISH is a fluorescent ISH, or FISH; and/or probes and/or reagents for conducting reverse transcription, or for converting RNA to a cDNA.

22. A method for assessing the gene breakage status of a gene of interest in a subject, the method comprising:
(a) performing an in situ hybridization analysis or an immunohistochemistry analysis on a biological sample from said subject to determine whether said subject has a gene breakage in said gene of interest; and
(b) if said in situ hybridization or immunohistochemistry analysis indicates that said subject has a gene breakage in said gene of interest or indicates that it is unclear whether or not said subject has a gene breakage in said gene of interest, sequencing or having sequenced at least a portion of a fused nucleic acid resulting from said gene breakage to identify the nucleic acid sequence fused to, or newly immediately adjacent to, said gene of interest.

23. The method of claim 22, wherein the gene breakage is a gene translocation, a gene rearrangement or a gene inversion.

24. The method of claim 22, wherein the process (a) comprises performing an ISH (In Situ Hybridization), and optionally the in situ hybridization analysis comprises a fluorescence in situ hybridization (FISH) analysis.

25. The method of claim 24, wherein said fluorescence in situ hybridization analysis comprises hybridizing a plurality of differently labeled nucleic acid probes to said gene of interest, wherein said plurality of differently labeled nucleic acid probes yield signals of a distinct first and second color if said gene of interest has undergone a translocation, and a signal of a third color resulting from the combination of said first and second color if said gene of interest has not undergone a translocation.

26. The method of claim 22, wherein said sequencing or having sequenced comprises generating a cDNA from a transcript of said fused nucleic acid.

27. The method of claim 26, wherein the sequencing or having sequenced comprises amplifying said cDNA and sequencing or having sequenced nucleic acids produced by said amplification.

28. The method of claim 27, wherein the sequencing comprises performing a PCR.

29. The method of claim 28, wherein the PCR comprises a multiplex PCR (mPCR), to amplify the gene of interest fused to one or more potential fusion partners or portions of the gene of interest fused to portions of one or more potential fusion partners.

30. A therapeutic method comprising: assessing the translocation state of a gene of interest using a method of claim 1; and if said gene of interest has undergone a translocation, administering a therapeutic agent or commencing with a therapy which is selected: based upon the finding of the identity of the nucleic acid fused to said gene of interest; or, based on characterization of the gene or genes involved in the gene breakage.
